(19) United States
(12) Patent Application Publication
(10) Pub. No.: US 2009/0164133 A1
(43) Pub. Date: Jun. 25, 2009

(54) INFORMATION MANAGEMENT TECHNIQUES FOR METABOLISM-RELATED DATA

(75) Inventor: Matej Oresic, Espoo (FI)

Correspondence Address: YOUNG & THOMPSON, 209 Madison Street, Suite 500, ALEXANDRIA, VA, 22314, US

(73) Assignee: Valtion Teknillinen Tutkimuskeskus, Espoo (FI)

(21) Appl. No.: 12/300,299

(22) PCT Filed: May 9, 2007
(86) PCT No.: PCT/FI07/50261

§ 371 Date: Feb. 25, 2009

(30) Foreign Application Priority Data
May 10, 2006 (FI) 20065309

(51) Int. Cl. G01N 33/48; H01J 49/26; G06F 19/00

(52) U.S. Cl. 702/19; 250/282

(57) ABSTRACT

A method for processing information on compounds of molecular classes sharing common building blocks. The method comprises maintaining pathway information on the compounds at individual compound level and/or generic class level (13-1); generating a diversity of the compounds based on a set of seed structures, each seed structure describing a lipid compound having a higher-than-average likelihood to occur in nature (13-2); using a formal description language to express the seed structures (13-3); using the structural elements to generate expected spectra for each compound, by using known experimental conditions for mass spectrometry (13-4); performing one or more spectroscopy experiments to obtain compound information (13-5); and linking the obtained compound information to existing information on the molecular classes (13-6).

### BACKGROUND OF THE INVENTION

The invention relates to information management techniques for metabolism-related data, such as data relating to lipids and/or other molecular classes sharing common building blocks, such as glycans.

Lipids are an important and highly diverse class of metabolites having structural, energy storage and signalling roles. Lipid metabolism is recognized to play a central role in several diseases such as arteriosclerosis, diabetes, and Alzheimer's, to name but a few. Despite such importance, the bioinformatics strategies to take full advantage of modern analytical and informatics technologies have not yet been presented.

Lipids are a diverse class of biological molecules that play a central role as structural components of biological membranes, energy reserves, and as signalling molecules. Dysfunctions of lipid metabolism are related to several human diseases, including diabetes, Alzheimer's disease, arteriosclerosis, and infectious diseases. While lipid and metabolome research in general, over the past decades, was overshadowed by the progress of genomics, recent revived and burgeoning interest in lipids that triggered many new endeavours in lipid research illustrates their critical biological importance. Lipidomics as a field aims at a characterization of lipid molecular species and their biological roles with respect to the expression of proteins involved in lipid metabolism and function including gene regulation.

Several useful public resources exist representing various aspects of information on lipids, such as LIPID MAPS, Lipid Bank, LIPIDAT, CyberLipids; and Lipid Base. New consortia have been formed such as LIPID MAPS (Lipid Metabolites and Pathway Strategy), and other pioneering groups from Europe and Japan are working towards similar interests. The LIPID MAPS consortium introduced a nomenclature that enables to represent a lipid compound by a unique 12-digit identifier. Following the same system of classification and nomenclature suggested by LIPID MAPS consortium, the JCBL (Japanese Conference on the Biochemistry of Lipids) maintains a related database Lipid Base, which also maintains MS/MS fragment information in individual lipid species.

Recent advances in analytical methods, particularly liquid chromatography coupled to mass spectrometry (LC/MS) for the studies of lipids, along with improved data processing software solutions, demand comprehensive lipid libraries to afford system level identification, discovery, and subsequent study of lipids. Integrative studies combining multi-issue lipidomic profiles with other levels of biological information such as gene expression and proteomics, have been made possible due to such capabilities.

Currently available databanks, particularly databases such as LIPID MAPS and Lipid Bank, offer a necessary starting point for such explorations and a reference for validation of results. However, in context of high-throughput lipidomic profiling and systems biology studies, the currently available online resources face twofold challenge:

Because of the large amounts of information available from high-throughput lipidomics experiments, any database system has to be efficiently linked to the analytical platform generating the lipid profile data, as well as to a chemo- and bioinformatics system for compound identification and linking the information to other levels of biological organization to enable systems approaches.

Due to the diversity of lipids across different organisms, tissues, and cell types a large majority of relevant lipids have not been identified and any single database is unlikely to cover all possible lipids. Thus a need exists for a mechanism to facilitate discovery of new lipid species in biological systems from available data.

In addition, currently available pathway-level representation of lipids in databases, such as KEGG, is limited to pathway representation of generic lipid classes, ie, including mainly the head-group information, and omitting the fatty acid side-chain information, and as such lacks the level of detail that is becoming available via LC/MS approaches. Current databases are very really useful as regards automated identification of a very large number of peaks. Thus a need exists for ways to identify individual molecular species. A related problem is how to link individual molecular species to the known metabolisms. For example, current pathway databases information only on generic lipid classes, such as pathways including phosphocholine. But there is no information on the underlying fatty acids. As a result, there can be hundreds of different species for phosphocholine.

Yet another related problem is how to generate lipid compounds diversity using information technology.

### BRIEF DESCRIPTION OF THE INVENTION

An object of the invention is to develop a method, an apparatus and software products so as to alleviate one or more of the problems identified above. The object is achieved with a method, an apparatus and software products which are defined by the appended independent claims. The dependent claims disclose specific embodiments of the invention.

An aspect of the invention is a method for processing information on compounds of molecular classes sharing common building blocks. The method comprises:
maintaining pathway information on the compounds at individual compound level and/or generic class level;
generating a diversity of the compounds based on a set of seed structures, each seed structure describing a lipid compound having a higher-than-average likelihood to occur in nature;
using a formal description language to express the seed structures;
using the structural elements to generate expected spectra for each compound, by using known experimental conditions for mass spectrometry;
performing one or more spectroscopy experiments to obtain compound information;
linking the obtained compound information to existing information on the molecular classes.

In a representative application of the invention, the compounds of molecular classes sharing common building blocks comprises lipids, and later in this document, the invention will be described in the context of lipids.

The step of maintaining pathway information on lipid compounds is known per se. There are numerous bioinformatics approaches for maintaining pathway information, and a detailed description is omitted.

In one embodiment of the invention, the formal description language is SMILES, which is an acronym for Simplified Molecular Input Line Entry System. Construction of a particular lipid class may be based on a SMILES template of that class. First, a generic SMILES template is generated, manually, for instance. Then the fatty acid chain length is varied to create many or all possible compounds of that class in a given window of fatty acid chain length. For instance, a PERL language parser can be developed for varying the fatty acid chain length. Once a SMILES presentation of a compound is generated, the SMILES presentation can be converted into a canonical (=unique) SMILES presentation. Another interesting feature of this method relates to its ease of creating systematic names algorithmically.

In the step of using the structural elements to generate expected spectra for each compound, it is beneficial to use commonly used experimental conditions.

The existing information on lipids contains pathway information at individual compound level and/or generic class level. Alternatively or additionally, the existing information on lipids may contain co-regulation information with other compound information across different biological samples.

In one embodiment, the method further comprises linking information on an individual compound to information at other levels. The information at other levels may contain information on proteins or genes related to the metabolism or biological variation of the individual compound.

In one embodiment, the method further comprises utilizing information on individual compound levels and their variation within a specific compartment across different biological samples, such as cell type, tissue or organ, to discover dependencies between compounds across different compartments.

### BRIEF DESCRIPTION OF THE DRAWINGS

In the following the invention will be described in greater detail by means of specific embodiments with reference to the attached drawings, in which:

FIG. 1 illustrates an operating principle of the operation;

FIG. 2 illustrates a database schema for representation of lipids;

FIG. 3 illustrates a method for systematic construction of glycerophospholipids;

FIG. 4 illustrates a technique for representing the structure of lipid compounds by using SMILES;

FIG. 5 illustrates a SMILES template showing fatty acid seed variables;

FIG. 6 illustrates structures of glycerophospholipids;

FIG. 7 illustrates an exemplary name template for glycerophospholipid class;

FIG. 8 illustrates a technique for using structural elements in the linking step;

FIGS. 9A and 9B, which form a single logical drawing, illustrate algorithmically constructed SMILES for an exemplary set of fatty acid chains;

FIGS. 10A and 10B illustrate generation of characteristic MS/MS spectra for individual species;

FIG. 11 illustrates a scoring system;

FIG. 12 illustrates how cross-tissue association study of lipid profiles at individual molecular species level across different organisms can reveal dependencies between biological processes in different compartments of the organisms; and

FIG. 13 summarizes the steps of a method according to the invention.

### DETAILED DESCRIPTION OF SPECIFIC EMBODIMENTS

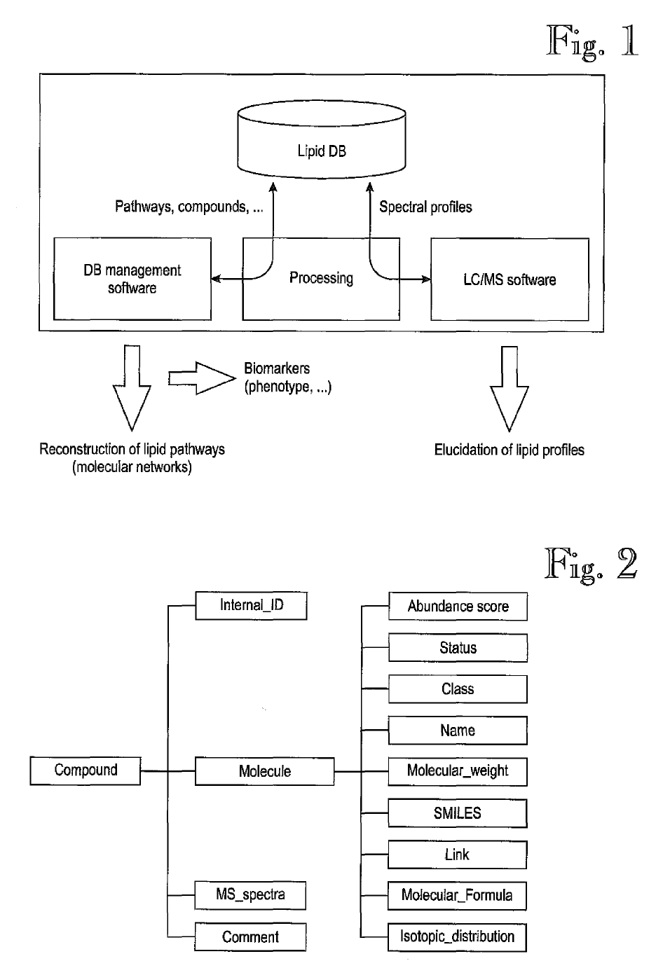

FIG. 1 illustrates an operating principle of the operation. Reference numeral 100 generally denotes a data system architecture in which the invention can be used. A lipid database 102 is accessed by a database management block 104 and a spectroscopy software block 106 via a processing block 108. In this implementation, the spectroscopy software block supports liquid chromatography/mass spectrometry, but other technologies can be used as well. The primary added value of the data system 100, over prior art systems, is twofold. Improved reconstruction of lipid pathways (eg molecular networks) is provided by the database management block 104 and improved elucidation of lipid profiles is provided by the spectroscopy software block 106.

Further details on how to construct an appropriate database management block 104 is disclosed by commonly assigned Finnish patent application FI20055198, titled “Visualization technique for biological information”, filed 28 Apr. 2005. In one aspect, the invention discosed by said commonly assigned application is a method for visualizing biological information, the method comprising: 1) generating a user interface and receiving a user query relating to biological information via the generated user interface; 2) maintaining connections to a plurality of databases which store at least partially non-overlapping biological information; 3) determining which database of the plurality of databases contains the biological information relating to the received query; 4) sending a database query to the determined database and receiving a result of the database query, the result comprising biological and/or chemical entities and relations between the biological and/or chemical entities; 5) creating a network based on the result of the database query, wherein the network-creating step comprises mapping the biological and/or chemical entities to network nodes and the relations to network connections; 6) determining a distance matrix for indicating a distance for several pairs of network nodes, each distance being calculated across several dimensions; 7) applying a dimensionality reduction function to map the distance matrix to a lower number of dimensions; 8) searching for neighbours of a selected network node based on the distance matrix in order to elucidate a biological role of the selected network node; 9) adjusting the dimensionality reduction function based on one or more research contexts of the biological and/or chemical information, in order to bias the search toward a relevant focus; and 10) re-creating and visualising the network based on the adjusted dimensionality reduction function.

The method may further comprise mapping each of the one or more research contexts to a network node and/or combining results of multiple database queries to different databases into a single network. The mapping step may comprise mapping from several dimensions to two dimensions. The distance function may be based on network topology and/or on relationships from experimental data. Such relationships from experimental data may comprise a correlation measure.

Further details on how to construct an appropriate spectroscopy software block 106 are disclosed by commonly assigned Finnish patent applications FI20055252, FI20055253 and FI20055254, all titled “Analysis techniques for liquid chromatography/mass spectrometry” and filed 26 May 2005. In one aspect, the invention discosed by said commonly assigned application FI20055252 is a method for analyzing liquid chromatography/mass spectrometry [=“LC/MS”] data, the method comprising: 1) preparing a plurality of sample runs; 2) processing each of the prepared sample runs in an LC/MS spectrometer to obtain a spectrum in respect of each processed sample run; 3) internally representing each spectrum as a layout of mass/charge versus retention time; 4) performing a first peak detection to detect peaks of each spectrum; 5) internally aligning the detected peaks of each spectrum; and 6) performing a second peak detection to detect peaks missed in the first peak detection.

In one aspect, the invention discosed by said commonly assigned application FI20055253 is a method for analyzing LC/MS data, the method comprising: 1) preparing a plurality of sample runs; 2) processing each of the prepared sample runs in an LC/MS spectrometer to obtain a spectrum in respect of each processed sample run; 3) internally representing each spectrum as a layout of mass/charge versus retention time; 4) performing a first peak detection to detect peaks of each spectrum; 5) visualizing peaks of each spectrum, wherein the visualizing step comprises: 5a) mapping each peak to be visualized to a coordinate system in which a first coordinate indicates mass/charge ratio and a second coordinate indicates retention time; and 5b) assigning a specific visual attribute to each peak to be visualized.

In one aspect, the invention discosed by said commonly assigned application FI20055254 is a method for analyzing LC/MS data, the method comprising: 1) preparing a plurality of sample runs; 2) processing each of the prepared sample runs in an LC/MS spectrometer to obtain a spectrum in respect of each processed sample run; 3) internally representing each spectrum as a layout of mass/charge versus retention time; 4) performing a first peak detection to detect peaks of each spectrum; and 5) searching for the standard compound peak closest to a peak being analyzed and normalizing the peak being analyzed based on a distance measure of the distance between the peak being analyzed and said closest standard compound peak.

The techniques for analyzing LC/MS data may be further enhanced by additional features. For example, the second peak detection may comprise detection of local maxima and/or recursive threshold detection. The method may further comprise normalizing the spectra, for example by injecting one or more standard compounds with a predetermined concentration into each sample run prior to the processing step in order to obtain a set of standard compound peaks for each injected standard compound. The method may further comprise searching for the standard compound peak closest to a peak being analyzed and normalizing the peak being analyzed based on a distance measure of the distance between the peak being analyzed and said closest standard compound peak. The aligning step may comprise generating a peak list in respect of each spectrum, generating a master peak list and for each peak in each peak list, finding the corresponding peak in master peak list by using a predetermined distance measure. The distance measure may be based on a weighted combination of |m/zₚ−m/zₘ | and |rtₚ−rtₘ|, wherein m/zₚ and rtₚ and are the mass-to-charge ratio and retention time, respectively, of a peak in an individual peak list, and m/zₘ and rtₘ are the average m/z ratio and retention timer respectively, of all peaks from different peak lists assigned to same row of the master peak list. The method may further comprise visualizing peaks of each spectrum, wherein the visualizing step comprises mapping each peak to be visualized to a coordinate system in which a first coordinate indicates mass/charge ration and a second coordinate indicates retention time; and assigning a specific visual attribute to each peak to be visualized. The visualization method may further comprise visualizing peaks from a first group and a second group of samples, and the specific visual attribute is based on a ratio of average intensities of corresponding peaks in the first group and a second group. Yet further, the visualization method may comprise visualizing peaks from a group of samples, and the specific visual attribute is based on a variation of peak intensities within the group of samples.

FIG. 2 illustrates an illustrative database schema for representation of lipids. In this illustrative but non-restrictive embodiment, lipid data is stored in a native XML database implemented in Tamino XML Server. Each compound entry in the database contains information about an internal identifier, scoring information, class, canonical SMILES, molecular formula, molecular weight and isotopic distribution. PERL scripts may be used to convert the data into XML documents. The resulting XML documents are loaded using mass-loading tool of the Tamino database. For the construction and validation of logical and physical schemas, XMLSPY software and Tamino Schema Editor Software, respectively, may be used. The Tamino XML Server and Schema Editor Software are available from Software AG, Germany. XMLSPY software is available from Altova, Inc.

Lipids are a diverse group of molecular species broadly defined as hydrophobic or amphipathic small molecules that may originate entirely or in part by carbanion based condensation of thioesters, and/or by carbocation based condensation of isoprene units. In this description of specific embodiments, the primary focus will be on establishment of informatics methods for studies of glycerophospholipids, sphingolipids, glycerolipids, and sterol esters.

The main structural variant among the above classes is variation within one or more fatty acid chains composing the lipid molecule. For example, glycerophospholipids can be represented by a few head groups such as choline or ethanolamine, while the diversity of possible fatty acid combinations and modifications attached to the functional groups is much higher.

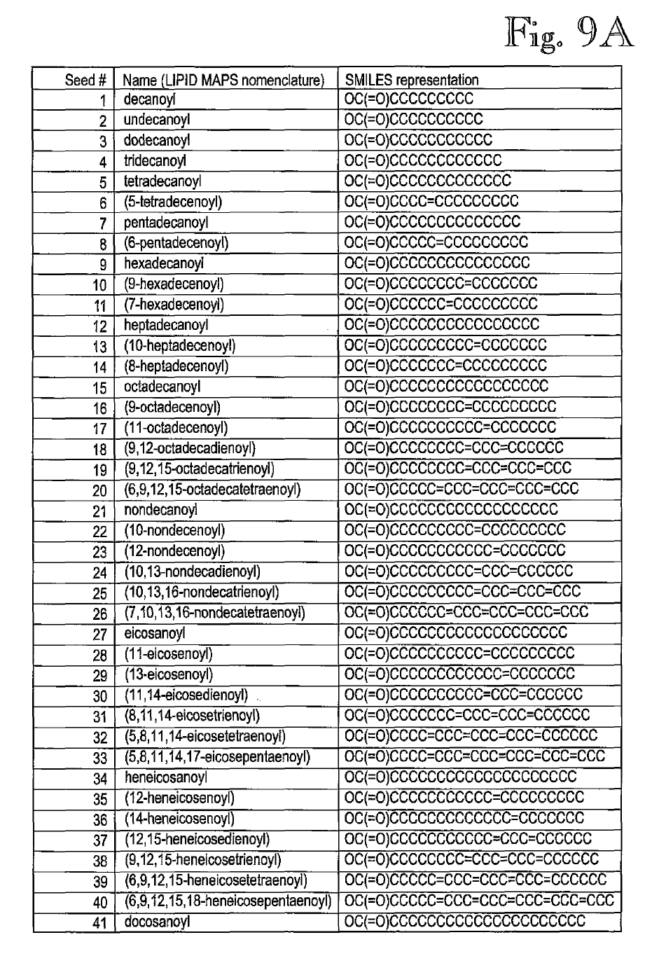

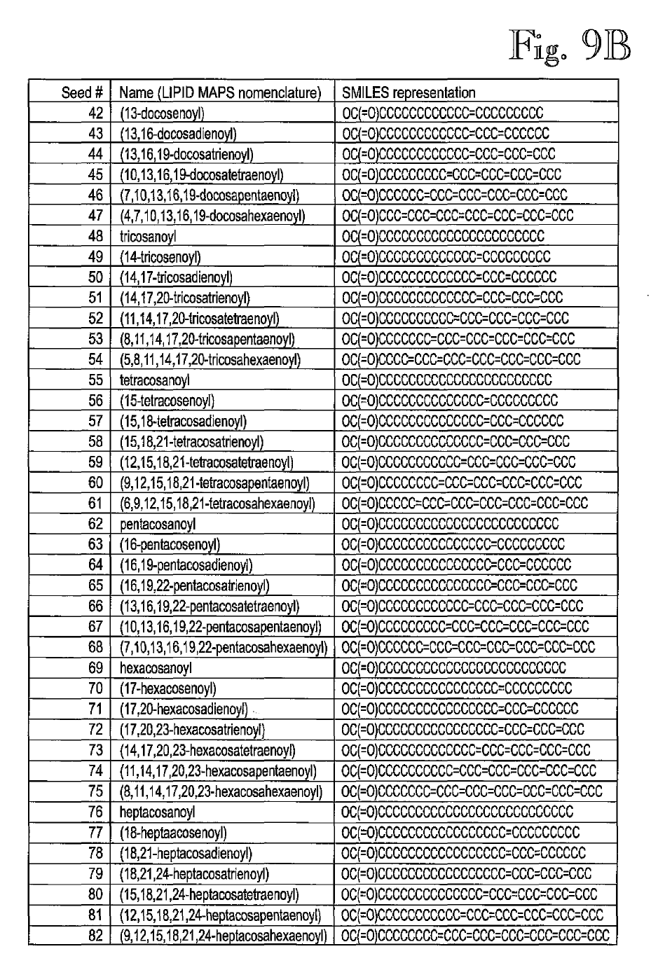

An advantageous approach to generating a diverse set of lipids to facilitate identification from lipidomics experiments is to generate a set of “seed” fatty acids most likely to occur in living systems. The choice of seed fatty acids described herein reflects a bias toward mammalian cells, but the inventive technique is flexible to addition of other fatty acids and functional groups. The fatty acid seeds are expressed in terms of Simplified Molecular Input Line Entry System (SMILES), which is a human readable linear indexing system of atoms and bonds, dictated by specific syntax rules. While in general multiple SMILES representations can exist for any given compound, canonical versions that enable unique SMILES representation are available. The embodiments described herein can be implemented by means of Daylight canonical SMILES representation (Daylight, Chemical information system, Inc.). SMILES have been constructed algorithmically for all these seed fatty acid chains and will be shown in FIGS. 9A and 9B. Systematic names adopted by LIPID MAPS consortium are used in constructing the lipid database.

A scoring value may be assigned to each compound in the database based on natural abundance of fatty acids from which that compound is formed. Common factors considered while assigning the scoring are natural abundance of the fatty acid and odd or even number of carbon atoms present in a fatty acid chain. In addition, different bindings of fatty acids to the lipid head group get different scores. The scoring system is illustrated in FIG. 11. The total score is then a product of all fatty acid scores.

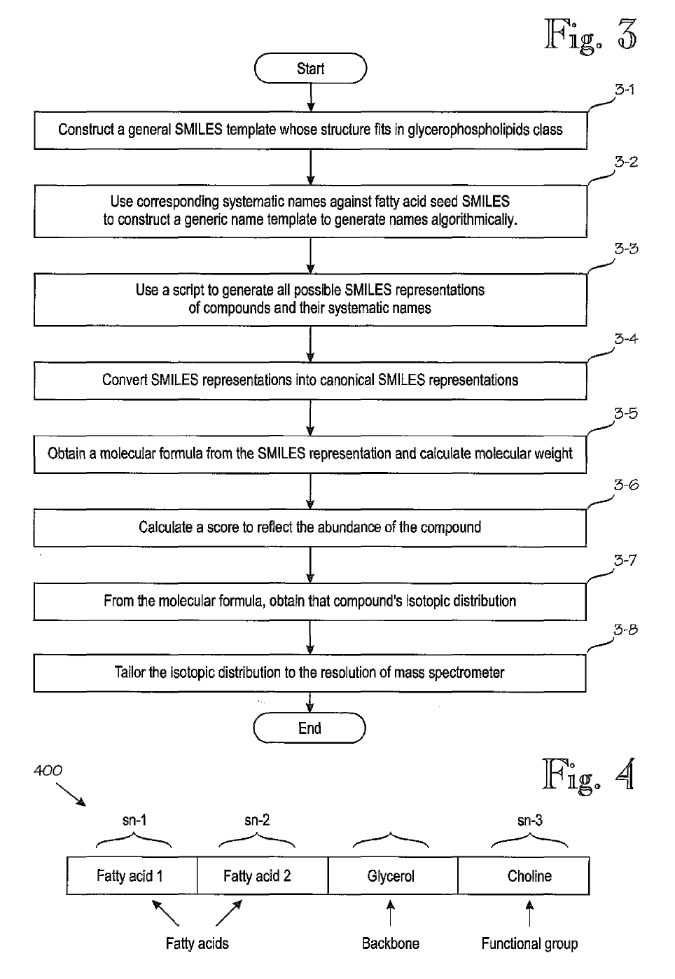

Construction of a particular lipid class is based on SMILES template of that class. Once a generic SMILES template is generated manually, PERL parsers may be developed for varying fatty acid chain length to create all possible compounds of that class in the given window of chosen fatty acid chain length. Once a SMILES of a compound is generated, one can convert SMILES into canonical (unique) SMILES. Another interesting feature of this method is about its ease of creating systematic names algorithmically. Daylight's SMILES tool kit may be used to generate canonical SMILES. Daylight toolkit has been tailored to get molecular weight and exact masses of compounds. Accurate masses of elements are taken from standard literature. A method for systematic construction of glycerophospholipids by using SMILES method is summarized in FIG. 3.

FIG. 11 illustrates a scoring system. A scoring value is assigned to each compound in the database based on natural abundance of fatty acids from which that compound is formed. Common factors considered while assigning the scoring are natural abundance of the fatty acid and odd or even number of carbon atoms present in a fatty acid chain. In addition, different bondings of fatty acids to the lipid head group get different scores. The total score is then a product of all fatty acid scores. Random score S of any lipid compound with fatty acid chains whose score variables Vi (at Sn1 position), V_{J} (at Sn2 position) and Vₖ (at Sn3 position) is obtained as follows. For compounds with single fatty acid chain at Sn1 or Sn2 position:

S=Vᵢ or Vⱼ

For compounds with two fatty acid chains at Sn1 and Sn2 positions:

=Vᵢ×Vⱼ

For compounds with three fatty acid chains at Sn1, Sn2 and Sn3 positions:

=Vᵢ×V_{J}×V_{K}

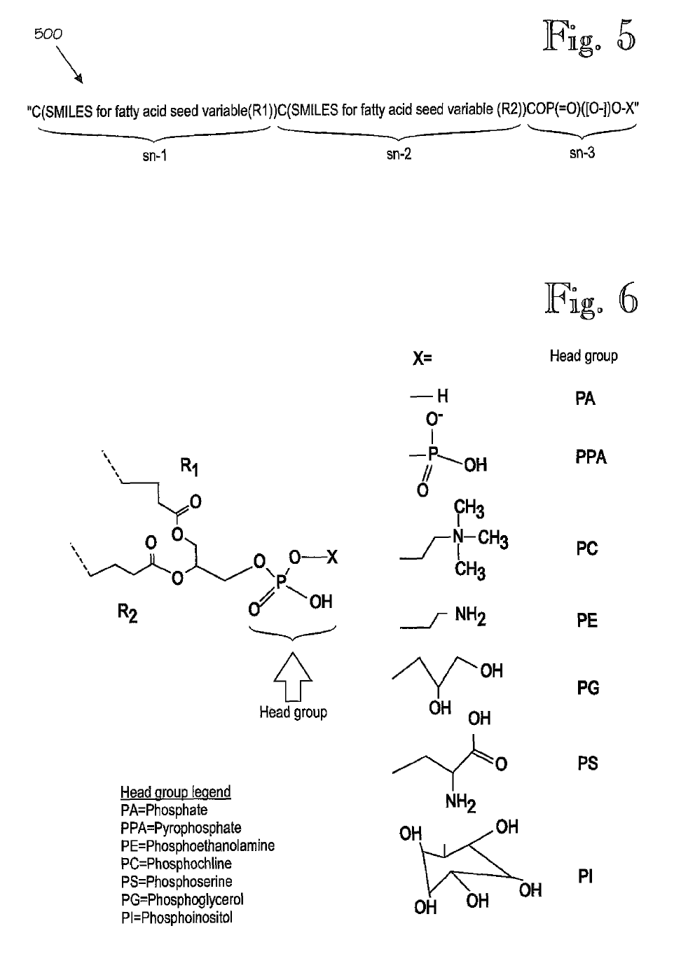

FIG. 3 illustrates a method for systematic construction of glycerophospholipids. Step 3-1 comprises construction of a general SMILES template whose structure fits in glycerophospholipids class. A SMILES template showing fatty acid seed variables for the sn-1 and sn-2 positions and head group variable (represented by symbol X) at sn-3 position (according to SMILES syntax rules, fatty acid seed variables are written in parenthesis representing them as branched chains) will be shown in FIG. 5. A set of appropriate structures will be shown in FIG. 6.

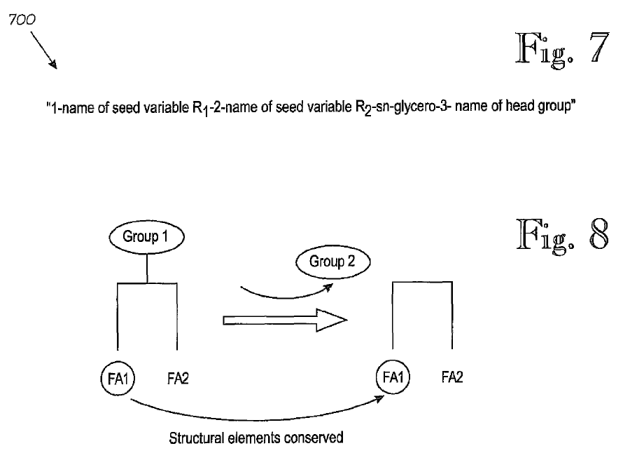

Step 3-2 comprises using corresponding systematic names against fatty acid seed SMILES to construct a generic name template to generate names algorithmically. An exemplary name template for glycerophospholipid class is shown in FIG. 7. An exemplary name table for retrieving systematic names is shown in FIGS. 9A and 9B.

Step 3-3 comprises use of a PERL script that generates all possible SMILES of compounds and their systematic names. Step 3-4 comprises conversion of SMILES into canonical SMILES (eg by using daylight SMILES toolkit). Step 3-4 comprises obtaining a molecular formula from SMILES and calculating the molecular weight for the obtained molecular formula. In step 3-6, a random score is calculated to reflect the abundance of the compound. In step 3-7, an isotopic distribution is obtained from the molecular formula of that compound. In step 3-8 the isotopic distribution is tailored to the resolution of the mass spectrometer to be used.

Spectral representation can be used together with LC/MS-based screening. To ease the identification of lipids based on the mass spectrometric data, isotopic distribution may be calculated for every compound in the database. This isotopic distribution may be based on observed natural abundance of each element in the chemical formula. Isotopic masses and abundances of given chemical composition are predicted using appropriate software, an example of which is open source Isotope Pattern Calculator. This theoretically generated distribution is very useful for comparison of isotopic patterns from mass spectrometric data. However, distributions obtained from mass spectrometer depend on its resolution. A PERL script may be used to convert calculated distribution to the desired distributions as per the resolution. Distributions can be displayed graphically.

The following description relates to generation of lipid compound diversity. The fact that these fatty acid chains remains as part of most lipid structures makes it possible to construct lipid classes algorithmically. The differences in length and degree of unsaturation in fatty acyl/alkyl chains create large diversity in a particular class itself. The lipid database may contain main classes such as fatty acyls, glycerolipids, glycerophospholipids, sphingolipids and sterols. Fatty acyls class includes fatty alcohols, fatty aldehydes, fatty carboxylic acids, fatty acyl CoAs/ACPs and eicosanoids. Glycerolipids class is relatively huge class in this database and contains sub classes such as mono acyl/alkyl glycerols, diacyl/alkyl glycerols and triacyl glycerols. The number of permutations of fatty acyl/alkyl chains at the three positions of glycerol, namely sn-1, sn-2 and sn-3, makes this class of compounds very huge. Glycerophospholipids is another important class and contains glycerolphosphocholines, glycerophosphoethanolamines, glycerophosphoserines, glycerophosphates, glyceropyrophosphates and glycerolphosphorglycerols. These compounds include both mono and diacyl/alkyl glycerolphospholipids. Plasmologens are special class of phospholipids where fatty acid chain of glycerol contains O-alkenyl ether (—O—CH═CH—) bonds. In one embodiment, the size of plasmologens subclass is 181548. Spingolipids class includes sphingoid bases, various ceramides including ceramide phosphorinositols, ceramide phosphocholines, ceramide phosphoethanolamines, N-acylsphingosines, N-acylsphinganines, ceramide 1-phosphates and sulfatides. In sterols, cholesteryl esters type compounds are present.

The lipid database mostly contains all possible lipids whose fatty acid chain lengths (or head groups if present in a class) can be varied algorithmically. One of the limitations of the SMILES method is that its difficulty in generating SMILES algorithmically for more complex lipids. For instance, complex lipids such as glycosphingolipids, whose SMILES are difficult to generate algorithmically, can be constructed manually. Another limitation with this database is redundancy. Lipids with same composition are difficult to distinguish. The problem of redundancy can be partially addressed based on the scoring values, since scoring sorts the redundant lipids according to their estimated frequency in nature. More common lipids get lower scores and vice versa. The scoring values are preferably adjusted for different organisms. Fragmentation and chromatography libraries are needed to address the issues of redundancy. The fragments of molecular ions corresponding to individual molecular species, preferably produced under different ionisation conditions, combined with retention time information from the reproducible analytical method, produce a unique signature of individual molecular species.

FIG. 4 illustrates a technique for representing the structure of lipid compounds by using SMILES. Reference numeral 400 generally denotes a structure of phosphocholine (PC). The phosphocholine structure 400 has fatty acids in the sn-1 and sn-2 positions, a glycerol backbone and choline in the sn-3 position. Like several lipids, phosphocholine is a class of molecules in which the fatty acids in the sn-1 and sn-2 positions can be varied to generate different phosphocholine compounds. Seed fatty acid are used, including common fatty acids, such as palmitic or oleic acids, etc., less common ones, such as odd-chain fatty acids,hydroxylated fatty acids, peroxides, etc.

FIG. 5 illustrates a SMILES template showing fatty acid seed variables for the sn-1 and sn-2 positions and head group variable (represented by symbol X) at sn-3 position. According to SMILES syntax rules, fatty acid seed variables are written in parenthesis representing them as branched chains).

FIG. 6 illustrates structures of glycerophospholipids with head groups such as phosphocholine (PC), phosphoethanolamine (PE), phosphoserine (PS), phosphoglycerol (PG), phosphoinositol (PI), phosphate (PA) and pyrophosphate (PPA).

FIG. 7 illustrates an exemplary name template for glycerophospholipid class.

FIG. 8 illustrates a technique for using structural elements in the linking step. Generally, a functional head group defines a lipid class. The conversion between different classes or their intermediates occurs at the level of the functional group, while the structural elements, such as fatty acids, which are specific to individual molecular species within a lipid class, are conserved.

FIGS. 9A and 9B, which form a single logical drawing, illustrate algorithmically constructed SMILES for an exemplary set of fatty acid chains.

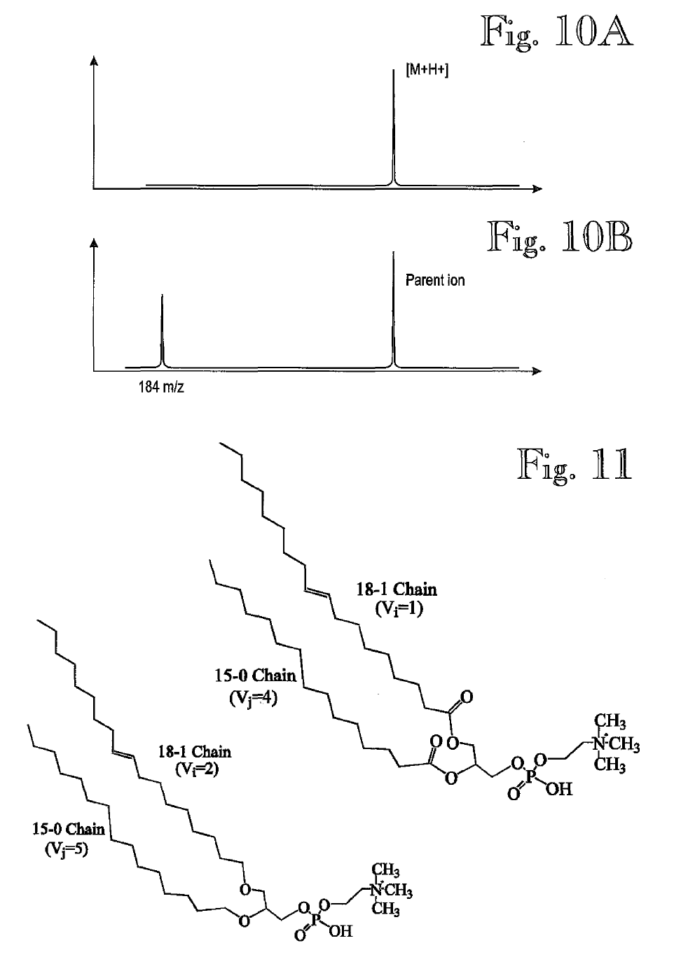

FIGS. 10A and 10B illustrate generation of characteristic MS/MS spectra for individual species. Using full scan MS method, following chromatography, the parent ion and retention time are recorded. The fragmentation of the parent ion, using MS/MS or similar methods, generates the fragments of the ion that, combined with information from MS and retention time, help elucidate the individual compound.

Techniques described in the above-specified Finnish patent applications FI20055252, FI20055253 and FI20055254 can be used to process spectral information that enters the searches for individual lipids. Techniques described in the above-specified Finnish patent application FI20055198 can be used to combine the lipid compound information with pathway information as well as with information at other biological levels.

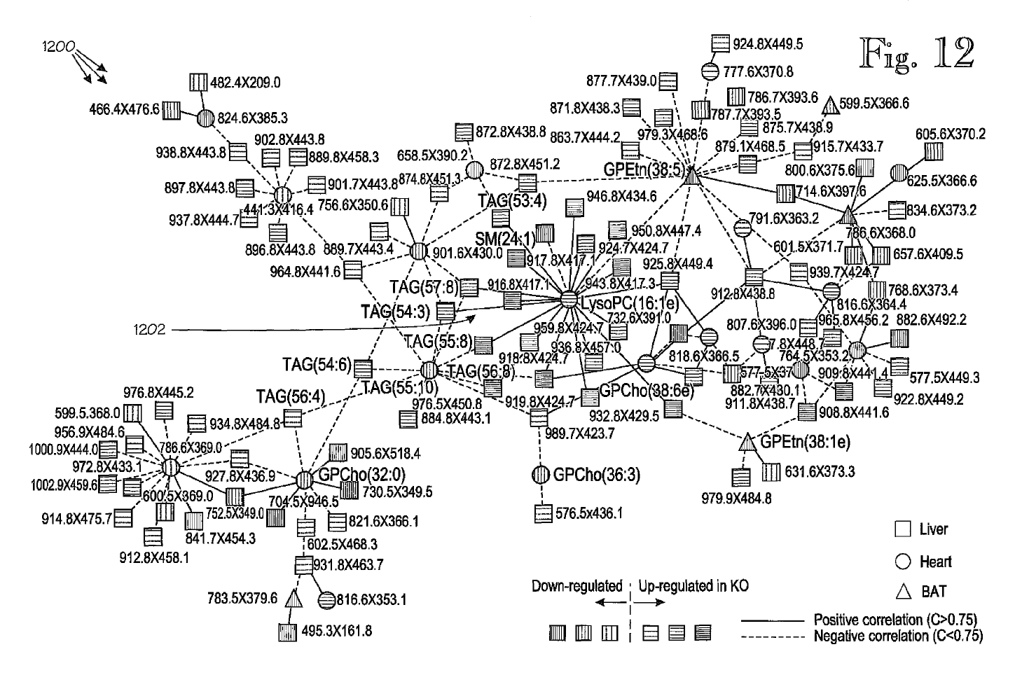

FIG. 12 shows a presentation of data 1200 which illustrates how cross-tissue association study of lipid profiles at individual molecular species level across different organisms can reveal dependencies between biological processes in different compartments of the organisms. The data 1200 shows a notable association of heart LysoPC (lysophosphatidylcholine) with liver TAGs (triacylglycerol), and negative association with BAT (brown adipose tissue) GPEtn (glycerophosphoethanolamine). For instance, an increase of specific triacylglycerols in liver is associated with increased ether-linked lysophosphatidylcholine in heart muscle, which is linked to the mitochondrial dysfunction in the heart. See eg the co-regulation between TAG 54:3 and LysoPC 16:1e. This co-regulation, denoted by reference numeral 1202, is a surprising discovery made by means of the method and data processing system according to the invention.

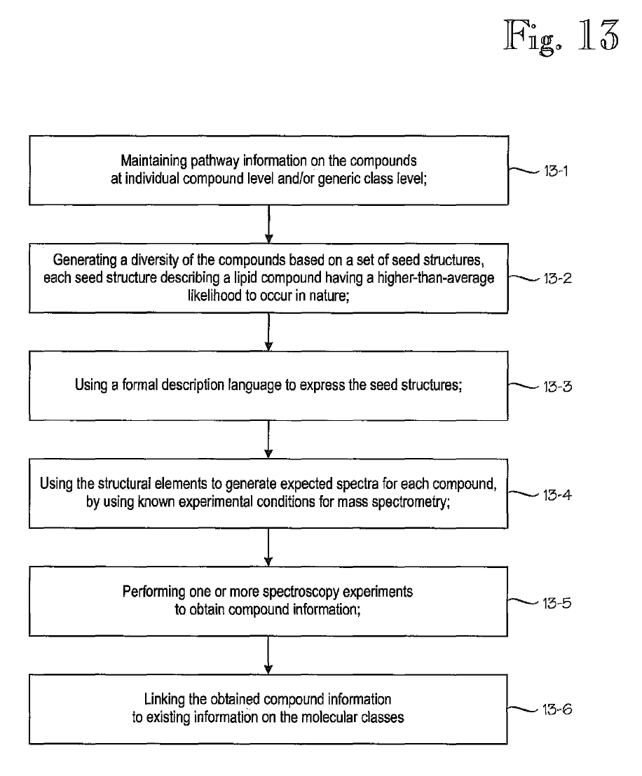

Finally, FIG. 13 summarizes the steps of a method according to the invention.

It is readily apparent to a person skilled in the art that, as the technology advances, the inventive concept can be implemented in various ways. The invention and its embodiments are not limited to the examples described above but may vary within the scope of the claims.

1-8. (canceled)

9. A method for processing information on compounds of molecular classes sharing common building blocks, the method comprising:
maintaining pathway information on the compounds at individual compound level and/or generic class level;
generating a diversity of the compounds based on a set of seed structures, each seed structure describing a lipid compound having a higher-than-average likelihood to occur in nature;
using a formal description language to express the seed structures;
using the structural elements to generate expected spectra for each compound, by using known experimental conditions for mass spectrometry;
performing one or more spectroscopy experiments to obtain compound information;
linking the obtained compound information to existing information on the molecular classes; and
outputting at least part of the linked compound information to a physical output device or storage device.

10. The method according to claim 9, wherein the existing information on the compounds contains pathway information at individual compound level and/or generic class level.

11. The method according to claim 9, wherein the existing information on the compounds contains co-regulation information with other compound information across different biological samples.

12. The method according to claim 9, further comprising linking information on an individual compound to information at other levels.

13. The method according to claim 12, wherein the information at other levels contains information on proteins or genes related to the metabolism or biological variation of the individual compound.

14. The method according to claim 9, further comprising utilizing information on individual compound levels and their variation within a specific compartment across different biological samples to discover dependencies between compounds across different compartments.

15. The method according to claim 9 wherein the compounds of molecular classes comprises lipids.

16. A data processing system for processing information on molecular classes sharing common building blocks, the data processing system comprising:
a database for maintaining pathway information on the compounds at individual compound level and/or generic class level; and
a processing logic for:
generating a diversity of the compounds based on a set of seed structures, each seed structure describing a lipid compound having a higher-than-average likelihood to occur in nature;
using a formal description language to express the seed structures;
using the structural elements to generate expected spectra for each compound, by using known experimental conditions for mass spectrometry;
performing one or more spectroscopy experiments to obtain compound information;
linking the obtained compound information to existing information on the molecular classes; and for
outputting at least part of the linked compound information to a physical output device or storage device.

17. The data processing system according to claim 16, wherein the existing information on the compounds contains pathway information at individual compound level and/or generic class level.

18. The data processing system according to claim 16, wherein the existing information on the compounds contains co-regulation information with other compound information across different biological samples.

19. The data processing system according to claim 16, further comprising linking information on an individual compound to information at other levels.

20. The method according to claim 19, wherein the information at other levels contains information on proteins or genes related to the metabolism or biological variation of the individual compound.

21. The data processing system according to claim 16, further comprising means for discovering dependencies between compounds across different compartments by utilizing information on individual compound levels and their variation within a specific compartment across different biological samples.

22. The data processing system according to claim 16 wherein the compounds of molecular classes comprises lipids.
